(19) United States
(12) Patent Application Publication
(10) Pub. No.: US 2024/0341798 A1
(43) Pub. Date: Oct. 17, 2024

(54) MULTIFUNCTIONAL HANDPIECE

(71) Applicants: Roberta MAZZANTI, Loiano (IT); Valentino GITTO, La Trinite’ (FR); Giorgio MAULLU, Oristano (IT); TARAS S.R.L., Bologna (IT); TEMA MEDICINA S.R.L., Bologna (IT)

(72) Inventors: Giorgio MAULLU, Oristano (IT); Tomaso Pietro GRAMOLI, Loiano (IT)

(21) Appl. No.: 18/681,362

(22) PCT Filed: Aug. 5, 2021
(86) PCT No.: PCT/IT2021/000041

§ 371 Date: Feb. 5, 2024

(51) Int. Cl. A61B 17/32; A61B 17/00

(52) U.S. Cl. CPC A61B 17/320068; A61B 2017/00084; A61B 2017/00747

(57) ABSTRACT

A multifunctional handpiece includes a main body provided with a proximal opening for the entry of a power supply and control cable, with control assembly, and with a distal opening for the connection of a stem; the handpiece further includes: at least one temperature sensor, arranged inside the body and a cable made of electrically conducting material; at least one ultrasound transducer connected to a sonotrode rigidly coupled to the stem. The stem is at least partially electrically conducting and includes, on a proximal part of the external surface, a cladding made of an electrically insulating material, and accommodates within its internal cavity the cable. A distal portion of the stem, without external cladding, is adapted for the transfer of radiofrequency energy to the tissues of a patient against which it is juxtaposed: the energy is adjusted by the external control and management unit electrically connected to the stem.

### CROSS-REFERENCE TO RELATED APPLICATIONS

This application is a 35 U.S.C. § 371 National Stage patent application of PCT/IT2021/000041, filed on 5 Aug. 2021, the disclosure of which is incorporated herein by reference in its entirety.

### TECHNICAL FIELD

The present disclosure relates to a multifunctional handpiece for the execution of endodermal treatments.

### BACKGROUND

Radiofrequency aesthetic/medical therapy utilizes the effect generated by adapted electromedical apparatuses, which produces a “remodeling” of tissues, with an action that is particularly effective in contrasting skin relaxation of the face and body.

The principle on which radiofrequency is based is the generation of a thermal shock in the deepest layers of the skin in order to trigger a regenerative response on the part of the body. The radiofrequency electromagnetic field is generated by a high-frequency (over 100 kHz) alternating electric current, the flow of which changes direction very rapidly and which does not stimulate the nervous and muscle tissue but has a controlled “thermal effect” due to the temperature increase.

Radiofrequency treatments can also be performed in endodermal mode.

By means of a specific needle cannula made of electrically conducting material and partially insulated superficially (connected to a main apparatus for control, management and power supply, capable of establishing the variable-frequency electrical field) it is possible to perform the radiofrequency treatment directly on the tissues of interest (under the skin of the patient).

This is possible by making the needle cannula pass through the dermis (or a predefined portion thereof) so that it can slide along a surface which is parallel to the skin of the patient, beneath it.

This type of treatment is used in aesthetic medicine in order to perform liposculpture operations on the patient, which allow to reduce (and even eliminate) imperfections, defects and other problems of a functional nature as well.

Other aesthetic medicine treatments, too, use endodermal rods, which however base their operating mode on principles that are completely different from radiofrequency.

Among these, it is known to resort to ultrasound treatments, in which a rod can be inserted below the dermis (through a specific incision): the front end of said rod, which constitutes the active element that vibrates at the frequency provided by the treatment, makes contact with the fat cells that are below the dermis and performs operations known as lipolysis and/or liposculpture.

Resorting to combined treatments has practically never been adopted in aesthetic medicine, since studies and research are aimed at a specific field of application, leading to a scientific prejudice against these options.

In any case, there are also problems of a technical nature which do not recommend to resort to the combination of radiofrequency and ultrasound treatments, since interference phenomena potentially dangerous for the instruments might be generated.

Furthermore, it should also be noted that the combination of different endodermal treatments currently is not possible, since it would be necessary to subject the patient to particularly invasive actions (a double incision for the entry of the cannula and of the rod or a continuous swapping between the cannula and the rod in the same skin incision).

### SUMMARY

The aim of the present disclosure is to solve the problems described above, devising a multifunctional handpiece which can be used equally to perform endodermal radiofrequency and ultrasound treatments.

Within this aim, the disclosure provides a multifunctional handpiece that can perform radiofrequency and ultrasound treatments simultaneously.
the disclosure also provides a multifunctional handpiece which comprises a needle cannula capable of dispensing energy according to the principles of radiofrequency treatments and according to the principles of ultrasound treatments.
the disclosure also proposes a multifunctional handpiece that is suitable to perform lipolysis and liposculpture treatments which are particularly effective and durable.
the disclosure further proposes a multifunctional handpiece that is particularly safe for the operator who uses it and for the patient subjected to the treatment.

A further advantage of the present disclosure is to provide a multifunctional handpiece that has a low cost, is relatively simple to provide in practice, and is safe in application.

This aim, as well as these and other advantages which will become better apparent hereinafter, are achieved by providing a multifunctional handpiece of the type comprising a main box-like body provided with a proximal opening for the entry of a power supply and control cable, with an optional control assembly, and with a distal opening for the connection of a substantially rigid hollow stem, characterized in that it comprises
at least one temperature sensor, arranged inside said box-like body, controlled by said control and management unit and comprising a cable made of electrically conducting material,
at least one ultrasound transducer connected to a sonotrode rigidly coupled to said rigid stem, said transducer being controlled by an external control and management unit,
and in that said rigid stem is at least partially made of electrically conducting material and comprises, on the proximal part of its external surface, a cladding made of an electrically insulating material, and accommodates within its internal cavity said cable, a distal portion of said stem, without external cladding, being suitable for the transfer of radiofrequency energy to the tissues of a patient against which it is juxtaposed, said energy being adjusted by said external control and management unit.

### BRIEF DESCRIPTION OF THE DRAWINGS

Further characteristics and advantages of the disclosure will become better apparent from the description of a preferred but not exclusive embodiment of the multifunctional handpiece according to the present disclosure, illustrated by way of non-limiting example in the accompanying drawings, wherein:

FIG. 1 is a partially sectional schematic perspective view of a possible embodiment of a multifunctional handpiece according to the disclosure and of the module connected thereto, and together they constitute an apparatus for medical/aesthetic treatments;

FIG. 2 is a schematic side view of a terminal portion of the handpiece of FIG. 1 without the hollow stem;

FIG. 3 is a schematic side view of the terminal portion of FIG. 1; and

FIG. 4 is an enlarged-scale schematic side view of a part of the terminal portion of FIG. 3.

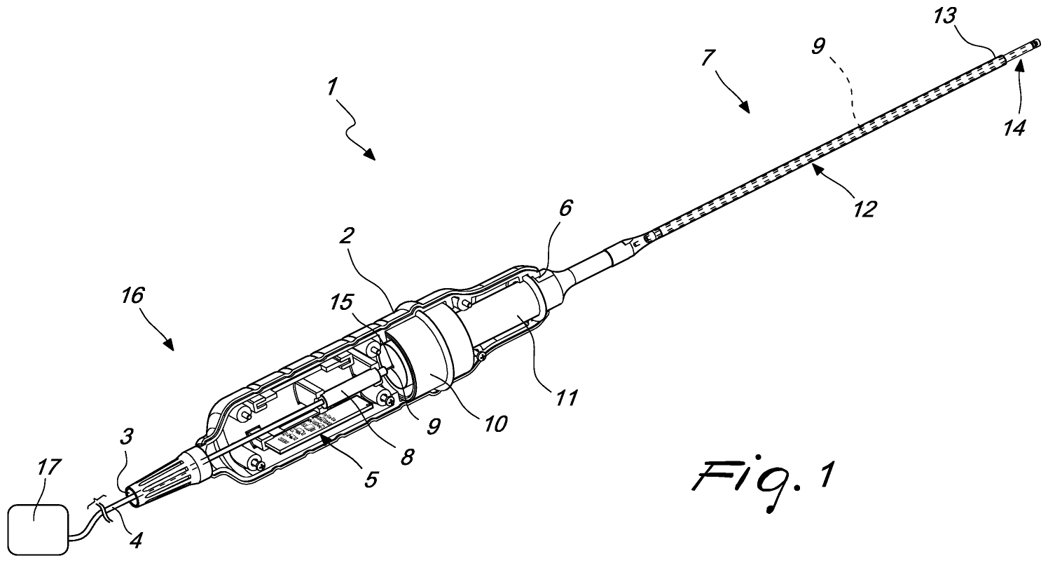

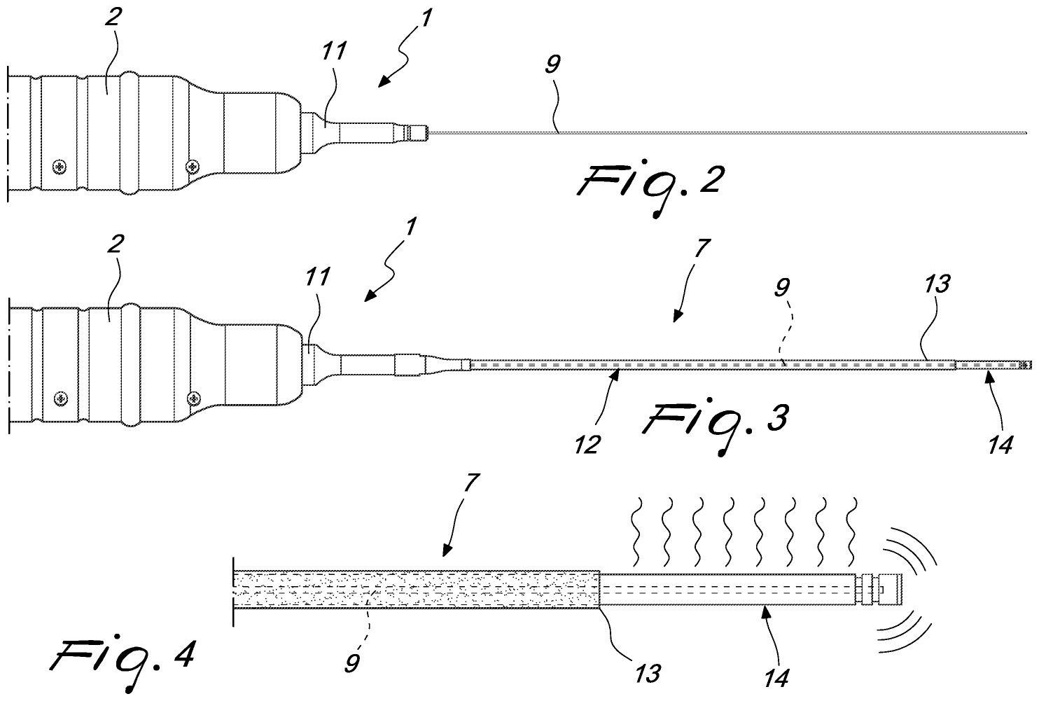

### DETAILED DESCRIPTION OF THE DRAWINGS

With reference to the figures, the reference numeral 1 generally designates a functional handpiece.

The multifunctional handpiece 1 according to the disclosure comprises a main boxlike body 2 provided with a proximal opening 3 for the entry of a power supply and control cable 4, with an optional control assembly 5, and with a distal opening 6 for the connection of a substantially rigid hollow stem 7.

The function of the (optional) control assembly 5 is to allow an external control and management unit (which comprises all the power stages of the power supply of the handpiece 1) to recognize the type and characteristics of the handpiece 1 associated therewith: this recognition allows the control and management unit to select the specific operating programs provided for each type of handpiece 1 associated therewith.

The handpiece 1 advantageously comprises at least one temperature sensor 8, for example of the type of a thermocouple or other similar sensor, arranged within the boxlike body 2, which is controlled by a control and management unit (which is external to the handpiece 1). Said external control and management unit, by means of an appropriate electric power supply, provides the power supply for the radiofrequency treatments (it comprises inside it a radiofrequency generator of a known type).

The temperature sensor 8 (for example a thermocouple) comprises a cable 9 made of electrically conducting material which protrudes from the distal opening 6 of the boxlike body 2 and reaches the stem 7: in practice, said sensor 8 is able to detect the temperature of a predefined portion of the stem 7 indeed by virtue of the specific length of the cable 9 (which allows the sensitive part of the sensor 8 to reach said portion of the stem).

The handpiece 1 furthermore conveniently comprises at least one ultrasound transducer 10 which is connected to a sonotrode 11 which is rigidly coupled to the rigid stem 7.

The transducer 10 also is advantageously controlled by the external control and management unit which, by means of an appropriate conditioning of its electric power supply, drives its operation.

It is noted that the rigid stem 7 is at least partially made of electrically conducting material and comprises, on the proximal part 12 of its external surface, a cladding 13 made of an electrically insulating material.

Furthermore, the rigid stem 7 may advantageously accommodate within its internal cavity the cable 9, which is in electrical and thermal contact with the internal surface of the cavity that accommodates it.

A distal portion 14 of the stem 7, without external cladding, is favorably suitable for the transfer of radiofrequency energy to the tissues of the patient of a patient that it faces and to which it is proximate: in particular, said distal portion 14 heats the tissues (of the patient) in contact with it (by means of the emission of radiofrequency electric power with a high current concentration at the distal portion 14, which induces heating by Joule effect) and can therefore be used for radiofrequency aesthetic medicine treatments in which the heating of the tissues of the patient firms them up and sculpts them.

In order to illustrate some possible specific constructive solutions of interest in application, it is specified that the ultrasound transducer 10 can advantageously be of the type suitable to generate vibrations with a frequency comprised between 16 kHz and 500 MHz and to transfer them to the sonotrode 11.

It is specified that the sonotrode 11 is of the type suitable to transmit vibrations with a frequency comprised between 16 kHz and 500 MHz to the stem 7.

The transducer 10, the sonotrode 11 and the stem 7 must be selected or designed in order to have common operating (or resonance) frequencies within the previously indicated ranges: the selection of specific frequency bands is dependent on the type of treatment for which the specific handpiece 1 is intended.

With particular reference to an embodiment of unquestionable interest in practice and in application, the transducer 10 and the sonotrode 11 can conveniently comprise a respective longitudinal channel 15, which allows the passage of the cable 9, so as to allow it to reach easily the cavity inside the stem 7 (up to the distal portion 14).

This constructive detail is extremely important, since ultrasound transducers 10 or sonotrodes 11 provided with through channels 15 are not particularly widespread commercially (in any case, these known channels are intended for deeply different purposes). The dimensions of the channels 15 must be calibrated as a function of the operating frequencies, so that they do not induce variations on the possible resonance and working frequencies of the components in which they are provided. Furthermore, the dimension must be such as to ensure the accommodation of the cable 9 with minimal transverse play, so as to prevent the vibrations from being able to induce unwanted movements of the cable 9 (which might cause its wear).

The possibility is provided to install a protective sheath on the cable 9 which also helps to increase its rigidity (an important characteristic when the stem 7 is removed for its sterilization, since it ensures that the cable 9 is more resistant to any unwanted external stresses).

In principle it is possible to assume that generally the cable 9 has a diameter of no more than 1 mm. The through channels 15 must therefore have a diameter that is larger than that of the cable 9: in percentage terms, the diameter of the channels 15 is greater than that of the cable 9 by between 2% (merely by way of example, in the case of a cable 9 with a diameter of 1 mm the channel would therefore have a diameter of 1.02 mm) and 100% (merely by way of example, in the case of a cable 9 with a diameter of 1 mm, the channel would therefore have a diameter of 2 mm). Adoption of the use of transducers 10 and sonotrodes 11 with channels 15 having a much larger diameter than the cable 9 for particular specific applications is not excluded.

With particular reference to an embodiment which is simple to use and extremely safe for the operator and for the patient, the sonotrode 11 can validly comprise a threaded terminal for the coupling of a proximal end of the stem 7, which also is provided with a thread which is complementary to that of said terminal.

By screwing the stem 7 to the terminal of the sonotrode 11, a very rigid and stable coupling is obtained which allows the perfect transfer of vibrations from the sonotrode 11 to the stem 7.

It is fundamental that these vibrations be transferred without attenuation, in order to ensure that the lipolysis or liposculpture treatment is effective.

From a constructive standpoint, it is specified that the stem 7 can conveniently be made of a material chosen from surgical steel, titanium, metallic alloys, composite materials, and the like.

The constructive requirement is that the stem 7 is at least partially electrically conducting: the transfer of radiofrequency energy to the tissues of the patient in fact occurs through the stem 7.

The control and management unit (which is external and not shown in the accompanying figures) comprises inside it a radiofrequency generator (of a type chosen between monopolar and bipolar) and, by means of appropriate conductors located inside the cable 4, it is possible to connect a terminal (or terminals) thereof to the stem 7.

In the case of a radiofrequency generator for monopolar treatments, only the hot pole of the generator is connected to the stem 7: this connection can occur by means of the electrical connection of said hot pole to the sonotrode 11 (rigidly coupled to the stem 7) or by means of the cable 9 which touches the surfaces of the cavity inside the stem.

In the case of bipolar treatments, both terminals of the radiofrequency generator are connected to the stem 7 (which must comprise specific insulations in order to separate the contracts related to the two distinct poles of the generator that are present on its surface).

In particular, it is also fundamental that the stem 7 be constituted by a thermally and electrically well-conducting material, so as to facilitate the transfer of radiofrequency electric energy (which will correspond to a high concentration of electric current transferred to the tissues) and the correct heating of the tissues (by Joule effect) which are skimmed by the distal portion 14. The distal portion 14 is heated by conduction (being in contact with the tissues of the patient which are heated by Joule effect) and by virtue of the cable 9, which constitutes a part of the thermocouple 8 (which comprises the bimetallic junction of said thermocouple 8, indeed at the distal portion 14), it is possible to detect instantaneously the temperature of said distal portion 14.

By means of the detection of the temperature of the distal portion 14 it is possible to control with feedback the quantity of energy transferred to the tissues in order to avoid a corresponding overheating, maintaining the ideal temperature chosen for the treatment that is being performed.

In general, all the materials described previously as possible components of the stem 7 are good electrical and thermal conductors.

It is specified furthermore that the cladding 13 can be constituted advantageously by a sheath made of polymeric material (with suitable biocompatibility characteristics) with high electrical resistivity, with dimensions suitable for covering all of the external surface of the stem 7 except for the distal portion 14 thereof.

The cladding sheath 13 can be extruded onto the stem 7 or deposited when it is in a viscous state (for example prior to cross-linking if it is made of a thermosetting polymer, or after being heated, if made of a thermoplastic polymer).

The adoption of a cladding sheath 13 made of heat-shrink material (which is fitted over the stem 7 and then heated to cause its contraction and locking) or elastomeric material (which is fitted over the stem 7 by utilizing its elastic deformability) is not excluded.

Furthermore, the interposition of adhesive substances between the cladding sheath 13 and the stem 7 in order to stabilize their arrangement is not excluded.

It is specified that the distal portion 14 without cladding can conveniently have an axial length, with respect to the longitudinal axis of the stem 7, comprised between 1 mm and 100 mm.

The specific length of the distal portion 14 will be assessed during design as a function of the type of treatment for which the handpiece 1 is intended.

The external control and management unit is conveniently suitable to deliver an electric current of the hot pole of the radiofrequency source that is adapted to produce the heating of the tissues of the patient that are in contact with the distal portion 14 of the stem 7 at a temperature comprised between 38° C. and 95° C. This electric current delivered by the hot pole of the radiofrequency generator has a frequency comprised between 100 kHz and 1 GHz.

The inventive concept of the present disclosure is also extended to an apparatus 16 for medical treatments which comprises a handpiece 1 in accordance with what has been described above: the handpiece 1 is connected to the main element of the apparatus by means of the cable 4.

The apparatus 16 comprises an external module 17 which accommodates said control and management unit as well as the radiofrequency generator that it controls.

If it comprises a radiofrequency generator for monopolar treatments, said apparatus 16 may furthermore comprise at least one conducting plate to be placed in electrical contact with a part of the skin of the patient, indeed according to the application criteria of radiofrequency treatments of the monopolar type.

In this manner, the distal portion 14 of the stem 7 constitutes a first branch of the electrical circuit which will close through the patient on the conducting plate, which in turn is connected to the apparatus in a return branch of the electric circuit.

As already mentioned previously, it is not excluded to provide a handpiece 1 that is suitable to perform radiofrequency treatments of the bipolar type (in which the two poles are both arranged on the stem 7, conveniently separated).

The external module 17 of the apparatus 16 can be provided with a specific interface for its user, which is constituted for example by a display, a screen, a speaker.

Said interface may also be provided remotely: the external module 17 of the apparatus 16 might in fact comprise signal transmission means (by means of a cable or wirelessly) with which to transmit the signals to a computer (provided with a respective screen or respective speakers), to a handheld computer, to a tablet, to a smartphone, to a smartwatch, to smart glasses, and the like.

The control and management unit (and the apparatus 16) determine many possible operating modes within the operating logic.

Energy according to two different principles, which correspond to radiofrequency medical treatments and to ultrasound medical and aesthetic treatments, can in fact be applied to said stem 7.

Ultrasound aesthetic medicine treatments have a mechanical effect on tissues, demolishing or breaking up at least partially the adipose accumulations by means of a respective “percussion”. The high vibration frequencies also cause a cavitation of some of the substances that make contact with the distal portion 14 of the stem 7, producing the lipolysis and liposculpture effects.

Endodermal radiofrequency medical treatments have a thermal effect which facilitates toning of the tissues and their contraction, which produces greater firmness thereof.

By virtue of the handpiece 1 according to the disclosure, during the steps of a lipolysis treatment (which are typical of ultrasound treatments) it is possible to perform a retraction/contraction of the tissues by means of a simultaneous (or alternating) endodermal radiofrequency treatment.

In practice the handpiece 1 according to the disclosure allows the aesthetic medicine operator to mix and/or alternate the two treatments.

Furthermore, the stem 7, even during an exclusively ultrasound treatment, can use the thermocouple 8 (associated by means of the cable 9 with the distal portion 14 that acts on the tissues of the patient) for a constant monitoring of their temperature (in order to ensure that the optimum conditions for the treatment being performed always occur).

In practice it is possible to dose the energy dispensed by means of the ultrasound as a function of the detected temperature, optimizing the quality of the treatment.

By virtue of the handpiece 1 it is therefore possible to utilize the synergistic effect (simultaneous or deferred, for example according to preset alternating sequences) of ultrasound treatments and radiofrequency treatments, but also to perform single treatments of a single type, having effective control of the temperature of the tissues of the patient, by means of which to adjust (with feedback) the energy parameters of said treatment.

Advantageously, the present disclosure solves the problems described above, proposing a multifunctional handpiece 1 which can be used equally to perform endodermal radiofrequency and ultrasound treatments in a mutual sequence and/or mutually alternated.

Conveniently, the multifunctional handpiece 1 can perform, even simultaneously, radiofrequency and ultrasound treatments.

Positively, the multifunctional handpiece 1 comprises a stem 7 that is capable of dispensing energy from its distal portion 14 according to the principles of radiofrequency treatments and according to the principles of ultrasound treatments.

Favorably, the multifunctional handpiece 1 is suitable to perform particularly effective and durable lipolysis and liposculpture treatments.

Advantageously, the multifunctional handpiece 1 is particularly safe for the operator who uses it and for the patient subjected to the treatment.

Validly, the multifunctional handpiece 1 is relatively simple to provide in practice and has low costs: these characteristics make the handpiece 1 and the apparatus 16 (which incorporates it) according to the disclosure innovations of assured application.

The disclosure thus conceived is susceptible of numerous modifications and variations, all of which are within the scope of the accompanying claims; all the details may furthermore be replaced with other technically equivalent elements.

In the examples of embodiment shown, individual characteristics, given in relation to specific examples, may actually be interchanged with other different characteristics that exist in other examples of embodiment.

In practice, the materials used, as well as the dimensions, may be any according to the requirements and the state of the art.

1-10. (canceled)

11. A multifunctional handpiece of the type comprising a main box body provided with a proximal opening for an entry of a power supply and control cable, with an optional control assembly, and with a distal opening for a connection of a substantially rigid hollow stem, and further comprising:
at least one temperature sensor, arranged inside said box body, controlled by said control and management unit and comprising a cable made of electrically conducting material,
at least one ultrasound transducer connected to a sonotrode rigidly coupled to said rigid hollow stem, said ultrasound transducer being controlled by an external control and management unit,
and wherein said rigid hollow stem is at least partially made of electrically conducting material and comprises, on a proximal part of an external surface thereof, a cladding made of an electrically insulating material, and accommodates within an internal cavity thereof said cable, a distal portion of said rigid hollow stem, without external cladding, being configured for a transfer of radiofrequency energy to tissues of a patient against which it is juxtaposed, said radiofrequency energy being adjusted by said external control and management unit.

12. The multifunctional handpiece according to claim 11, wherein said ultrasound transducer is configured to generate vibrations with a frequency comprised between 16 kHz and 500 MHz and to transfer said vibrations to said sonotrode.

13. The multifunctional handpiece according to claim 11, wherein said sonotrode is configured to transmit vibrations with a frequency comprised between 16 kHz and 500 MHz to said stem.

14. The multifunctional handpiece according to claim 11, wherein said ultrasound transducer and said sonotrode comprise a respective longitudinal channel for a passage of said cable.

15. The multifunctional handpiece according to claim 11, wherein said sonotrode comprises a threaded terminal for a coupling of a proximal end of said rigid hollow stem, provided with a thread which is complementary to a thread of said threaded terminal.

16. The multifunctional handpiece according to claim 11, wherein said rigid hollow stem is made of a material chosen from surgical steel, titanium, metallic alloys, and composite materials.

17. The multifunctional handpiece according to claim 11, wherein said cladding is constituted by a sheath made of polymeric material with high electrical resistivity with dimensions adapted to cover all of the external surface of said stem except for said distal portion of said rigid hollow stem.

18. The multifunctional handpiece according to claim 17, wherein said distal portion without cladding has an axial length, with respect to a longitudinal axis of said rigid hollow stem, comprised between 1 mm and 100 mm.

19. An apparatus for aesthetic treatments, comprising a multifunctional handpiece according to claim 11 and an external module which accommodates said external control and management unit and at least one radiofrequency generator of a type chosen from monopolar and bipolar.

20. The apparatus according to claim 19, wherein said external module, by means of said external control and management unit, is configured to deliver an electric current for a power supply of said rigid hollow stem which is adapted to determine a heating of the tissues of the patient that are in contact with said distal portion of said rigid hollow stem to a temperature comprised between 38° C. and 95° C., said electric current for the power supply of a thermocouple having a frequency comprised between 100 kHz and one GHz.
